(19) United States
(12) Patent Application Publication
(10) Pub. No.: US 2025/0223628 A1
(43) Pub. Date: Jul. 10, 2025

(54) METHODS OF IMPROVING SURVIVAL AND ELIMINATING TREATMENT-LIMITING TOXICITIES OF DICHLOROACETATE IN PATIENTS WITH PYRUVATE DEHYDROGENASE COMPLEX DEFICIENCY

(71) Applicants: SAOL INTERNATIONAL DEVELOPMENT LTD., Hamilton (BM); University of Florida Research Foundation, Incorporated, Gainesville, FL (US)

(72) Inventors: David PENAKE, Atlanta, GA (US); Sharon HAMM, Odessa, FL (US); Susan CLEMENT, Zionsville, PA (US); John DEVANE, Dublin (IE); Peter STACPOOLE, Gainesville, FL (US)

(73) Assignees: SAOL INTERNATIONAL DEVELOPMENT LTD., Hamilton (BM); University of Florida Research Foundation, Incorporated, Gainesville, FL (US)

(21) Appl. No.: 19/012,392

(22) Filed: Jan. 7, 2025

Related U.S. Application Data
(60) Provisional application No. 63/618,529, filed on Jan. 8, 2024.

(51) Int. Cl. C12Q 1/533; A61K 31/19; C12Q 1/6883

(52) U.S. Cl. CPC C12Q 1/533; A61K 31/19; C12Q 1/6883; C12Q 2600/172

(57) ABSTRACT

Methods of improving survival and eliminating treatment-limiting toxicities of dichloroacetate (DCA) in patients with pyruvate dehydrogenase complex deficiency (PDCD).

### STATEMENT OF GOVERNMENT SUPPORT

The invention was made with government support under grant numbers R01 HD087306 and R42 HD089804 awarded by the National Institutes of Health, and R01 FD007271 and R43 FD005349 awarded by the US Food & Drug Administration. The government may have certain rights in the invention.

### FIELD OF THE INVENTION

This invention relates to methods of improving survival and eliminating treatment-limiting toxicities of dichloroacetate (DCA) in patients with pyruvate dehydrogenase complex deficiency (PDCD).

### BACKGROUND OF THE INVENTION

Pyruvate dehydrogenase complex deficiency (PDCD) is a rare, genetic disorder of metabolism. Patients with PDCD experience a significant reduction in their ability to process sugars into energy through oxidative phosphorylation because the PDC complex is defective and therefore has a lower flux. This results in glycolysis becoming the primary source of energy production and leaves patients severely afflicted with high levels of lactic acid, developmental delay, organ malformations and early death.

PDCD is associated with high morbidity and mortality. Two natural history studies have been conducted evaluating the natural course of the disease. One study [DeBrosse, et al 2012] was conducted at a single site in a cohort of 59 patients. In-depth chart reviews and family interviews were conducted to establish the most common symptomology, different medical interventions and whether the patient remained alive or had passed away. 23 of the 59 patients in the study (39%) had passed away at the time of the publication and at least 2 additional patients passed away following a later review. Another study [Patel, et al 2012] conducted a global meta-analysis using data on published data that included subjects, primarily in case report form, with PDCD. This review included 371 patients, of which 294 reported survival outcomes at the time of publication. Of the 294, 108 patients (37%) had died. Both studies reported higher rates of mortality at younger ages. Patients that survived past one year of life had a higher likelihood of surviving in early childhood. If patients survived past 4 years of age, they had a much higher likelihood of surviving into adolescence. Despite improved lifespan, most patients had very severe mental and physical deficits that often made them completely dependent on care.

Dichloroacetate (DCA) is an investigational drug that has potential for the treatment of PDCD. DCA acts as a pyruvate dehydrogenase kinase inhibitor. By inhibiting pyruvate dehydrogenase kinase (PDK), which normally shuts off the pyruvate dehydrogenase complex (PDC) when a cell has enough ATP, DCA increases the production of energy by mitochondria.

DCA is metabolized almost exclusively in the liver by the GSTZ1 enzyme. This enzyme is down-regulated by the presence of DCA and is further down-regulated with age. Therefore, patients that are prescribed DCA will have a reduced ability to clear the medication each day until homeostasis is reached and will also have a reduced ability to clear it if they are older.

Additionally, studies have shown the GSTZ1 enzyme is expressed differently in different populations, which leads to differential metabolizer groups for DCA. It has been hypothesized that genetic testing for GSTZ1 haplotypes could lead to improved outcomes with DCA treatments for PDCD. [Langaee et al., 2015; U.S. Pat. No. 9,765,393.]

### SUMMARY OF THE INVENTION

The present invention is made on the basis of the extremely surprising finding that genetic testing for the GSTZ1 haplotype can not only improve outcomes, it can practically eliminate death as a side effect of DCA treatment.

The invention provides, among other features, methods of improving survival and minimizing treatment-limiting toxicities in dichloroacetate (DCA) treatment of patients with pyruvate dehydrogenase complex deficiency (PDCD) comprising: 1) identifying the DCA-metabolic haplotype of the patient, and 2) administering a DCA dose based on the DCA-metabolic haplotype.

In some aspects, the DCA-metabolic haplotype is identified by performing an assaying for the GSTZ1 haplotype in the patient.

In some aspects, the method reduces the likelihood of DCA-induced side effects to less than 10%, or to less than 5%, or to less than 2%. Such side effects include, but are not limited to, neuropathy.

In some aspects, the method reduces the likelihood of PDCD-related mortality to less than 10%, or to less than 5%, or to less than 2%. Reductions can also be to less than 9%, less than 8%, less than 7%, less than 6%, less than 4%, less than 3%, less than 1%.

In some aspects, the method reduces the odds ratio of death to less than 0.2%, to less than 0.10%, to less than 0.075%, or to less than 0.05%.

Other features and advantages of the present invention will be set forth in the description of invention that follows, and will be apparent, in part, from the description or may be learned by practice of the invention. The invention will be realized and attained by the devices and methods particularly pointed out in the written description and claims hereof.

### BRIEF DESCRIPTION OF THE FIGURES

FIG. 1 shows a Kaplan-Meier plot of percentage of survival of 36 subjects.

FIG. 2 shows results of the worm growth assay.

FIG. 3 shows results of the worm swim activity assay.

FIG. 4 shows results of the mitochondrial health assay.

FIG. 5 shows results of the worm lifespan assay.

FIG. 6 shows overall survival rates in the sex- and age-matched groups.

FIG. 7 shows overall survival by year of birth in natural history cohort.

FIG. 8 shows overall survival in the sex- and age-matched groups with extended follow-up time.

FIG. 9 shows overall survival in the sex- and age-matched groups, excluding patients born in the 1980s.

FIG. 10 shows overall survival in the age-matched groups.

FIG. 11 shows overall survival in the full cohorts.

FIG. 12 shows overall survival in the sex- and follow-up duration-matched groups.

### DETAILED DESCRIPTION OF THE INVENTION

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the recitation of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range.

DCA has been administered to hundreds of people for multiple indications. That breadth of exposure identified severe, reversible, peripheral neuropathy as a side-effect that limited continued development of DCA, despite a significant clinical interest in the target of the mechanism of action. In several studies across multiple disease states, ˜44% of patients experienced this side-effect that made the therapy intolerable. Recovery from peripheral neuropathy exhibited during DCA administration can take as long as 9-months, therefore dose reduction has not been shown to be a viable method of risk reduction.

| | | | Peripheral | Peripheral |
|---|---|---|---|---|
| | | | Neuropathy | Neuropathy |
| | | Total | Adverse | Adverse Event |
| Study | Disease | Patients | Events | Rate |
| Abdelmak 2013¹ | Congenital Lactic Acidosis | 8 | 3 | 38% |
| Michelakis 2017² | Pulmonary Arterial Hypertension | 20 | 5 | 25% |
| Dunbar 2014³ | Recurrent Malignant Brain Tumor | 8 | 2 | 25% |
| Stacpoole 2008⁴ | Congenital Lactic Acidosis | 36 | 13 | 36% |
| Kaufmann 2006⁵ | MELAS | 15 | 15 | 100% |
| Total | | 87 | 38 | 44% |

Several expert clinicians reviewed data on the patients that had participated in several of the studies and identified that different GSTZ1 haplotypes had a different risk in the likelihood of experiencing peripheral neuropathy and noted that the slow metabolizer group of haplotypes require roughly ½ the dose for a similar pharmacokinetic exposure. It has been hypothesized that this could reduce the risk of neuropathies to a tolerable level.

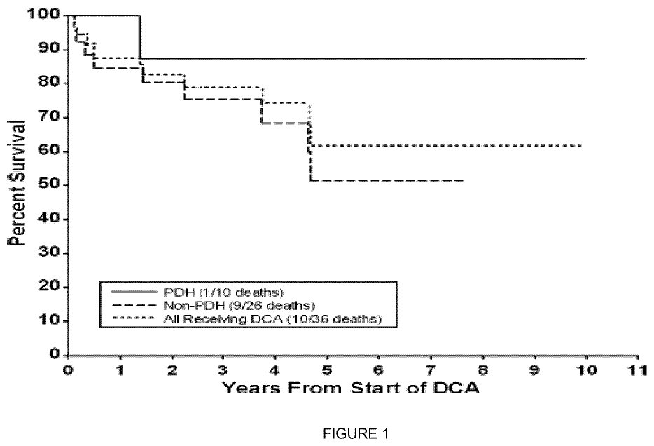

A randomized, double blind, placebo controlled study of patients with congenital lactic acidosis (CLA) of all causes in which patients were administered DCA at a dose of 25 mg/kg broken into two daily doses. 36% of the patients in this study double blind portion and 38% of the patients in the long-term extension arm experienced peripheral neuropathy. The long term outcomes of a subset of patients were evaluated. Of the 10 patients that had PDCD and adhered to chronic dosing of DCA, 9 of them experienced survival of up to 10 years. (See FIG. 1.)

Studies were also conducted in C. elegans using genetic knockdown technologies to create two PDCD disease models. The first was a model of PDHA1 knockdown worms. PDHA1 is the most common genetic defect that leads to PDCD and is associated with 80-85% of all PDCD cases [Patel 2012, Debrosse 2012]. This defect occurs at the E1 subunit of the PDC enzyme complex. Dihydrolipoamide (DLD) knockdown worms were also created. DLD is a form of PDCD that is caused by a defect at the E3 subunit of the PDC complex.

DLD knockdown worms had the most severe phenotype of the two genetic defects. Therefore, 3 severities of DLD knockdown worms were created, full dose, 1:20 dilution of DLD knockdown RNAi and a 1:100 dilution of DLD knockdown RNAi. The three DLD worm mutants and the PDHA1 mutant were tested by creating different concentrations of DCA in the water which they lived. The DCA concentrations were: 0 uM (water/control), 10 uM, 100 μM, 1 mM, 5 mM and 25 mM. Multiple measures of overall worm health and effect were measured. These included morphology and organ development, activity measures, mitochondrial stress and lifespan and biochemical measures of lactate. A dose dependent improvement in all measures was observed for the PDHA1 phenotype. Improvement (reduction) in mitochondrial stress was not observed at any DCA dose. Lifespan measurements were conducted on PDHA1, DLD 1:20 and DLD 1:100 worms at the 25 mM dose of DCA. Statistically significant improvements were observed in the PDHA1 and DLD 1:20 worms.

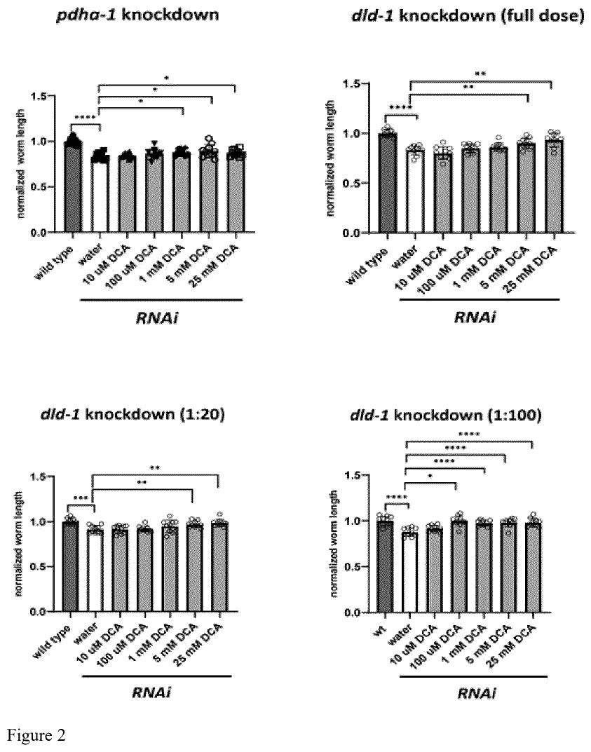

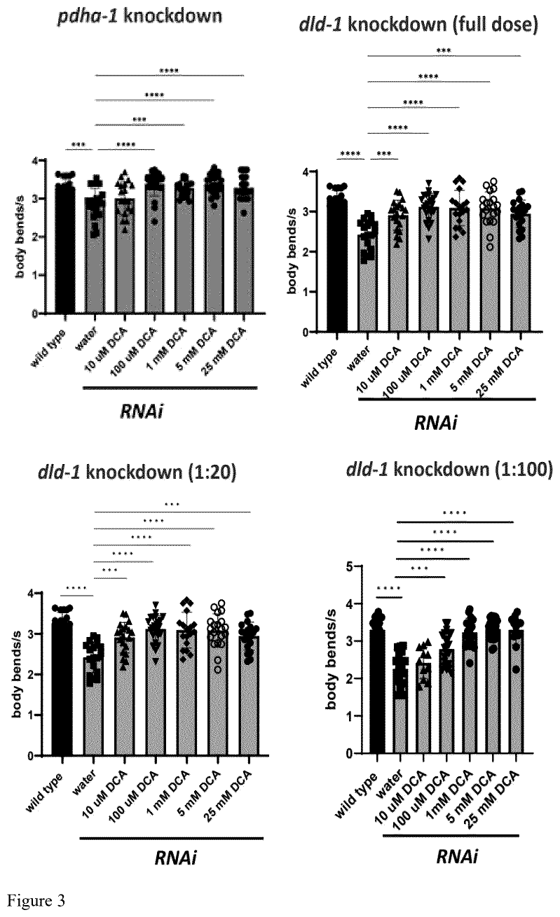

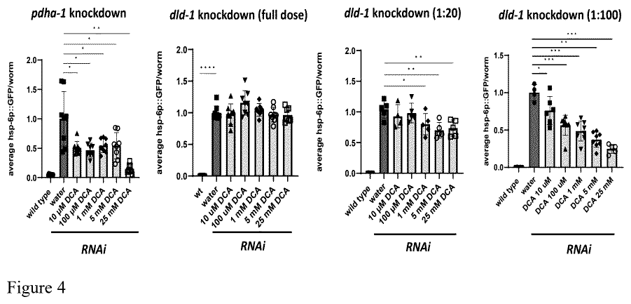

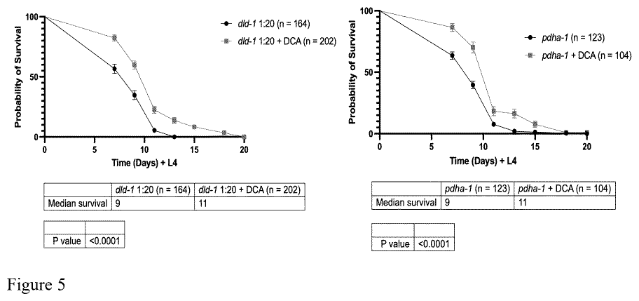

The results of the worm studies are shown in FIGS. 2-5.

Taken together, these results suggest that the clinical efficacy of DCA in PDCD is highly dose dependent. The higher the dose, the more likely that clinical benefit will be obtained and observed. As noted in the results, many of the most significant improvements were only obtained at the 25 mM dose which is well above common human exposure.

The therapy limiting toxicity of peripheral neuropathy is also dose-dependent with increasing risk occurring at increasing levels of DCA exposure. This raises the concern about whether therapeutic levels of DCA can be administered with an acceptable safety profile. It was plausible that the dose required for therapeutic benefit, particularly related to the extension of life, would require sufficiently high doses that would ultimately lead to peripheral neuropathy.

A prospective, randomized, double-blind, placebo-controlled crossover study was conducted in patients with PDCD. This study was the first study to prospectively use GSTZ1 haplotype testing to identify a dose that may help to mitigate the risk of peripheral neuropathy from chronic administration of DCA. These patients were treated with either DCA or placebo for 4 months, provided placebo for a 1-month washout period and then were placed on the placebo or DCA, whichever they had not taken prior. Patients then had the option to continue on open-label DCA until the ultimate completion of the study or regulatory approval. 34 PDCD patients were recruited into the study. 7 (21%) of these patients were prospectively identified as “slow metabolizers”. Rather than having a dose of 25 mg/kg/day, these patients were provided DCA doses at 12.5 mg/kg/day. Both dosing groups took DCA in two equal, weight-based doses. This was the first prospective study completed using the haplotype testing and conducted solely in the PDCD patient population.

Over the course of the study, it became surprising and notable that all of the patients completed the double-blind portion. The study had anticipated a 20% dropout rate due to death or peripheral neuropathy. Additionally, no observations of death were reported in the patients that entered the open label portion of the study at the time the last patient enrolled in the double-blind portion of the study.

As a result, an analysis procedure was identified that used the data available in the DeBrosse natural history study to assess whether or not DCA extended life. The analysis was conducted to answer the following questions:
a. Did the year of birth have any effect on survival? For example, has standard of care improved.
b. Is the primary driver of survival differences from natural history driven by the ketogenic diet, the current standard of care, although empirical approach to managing PDCD patients?
c. Once the population is controlled for an index age, gender and age of disease onset, does DCA provide a survival benefit?
d. Once the population is controlled for an index age and age of disease onset, does DCA provide a survival benefit?
e. Once the population has the maximum duration of follow-up, including with patients that discontinued treatment, does DCA provide a survival benefit?

Of the 59 patients (27 males and 32 females) identified by DeBrosse et al., 25 were excluded from eligibility in the study: six did not have an E1 subunit defect (DLAT or PDHB), six received DCA therapy, and 15 were <0.9 years at their last known age. After accounting for two patients with multiple exclusion criteria, 34 patients (14 males and 20 females) from the DeBrosse study remain for potential matching for the current analyses.

Baseline characteristics related to drug and non-drug treatment, age at the earliest recorded of first related symptom, initiation of the ketogenic diet or diagnosis and other baseline demographics were compared for each analysis. It was notable that the Phase III cohort had more females. This was controlled by conducting a 1:1 matched cohort in the primary analysis that requires gender matching.

The results of the study are as follows:
a. The year of birth did not have an effect on survival, p=0.4051 using log rank statistics.
b. The ketogenic diet did not have an effect on survival. This was analyzed as a covariate in each model and under no scenarios was shown to exhibit a positive impact on survival. In fact, life-span trended shorter in patients with the ketogenic diet.
c. DCA increased life-span in the 28 age and gender matched patients.
d. DCA increased life-span in the 32 age matched patients.
e. DCA increased life-span in 28 patients with duration follow-up
f. The only death observed was that of a patient that discontinued therapy for 6 months prior to the death (extended follow-up time analyses).

A summary of all analyses conducted with both Kaplan-Meier log-rank statistics and hazard/odds ratio reductions are provided below:

| | DCA Treatment Cohort | | Natural History Cohort | | | Adjusted | |
|---|---|---|---|---|---|---|---|
| | | Mortality | | Mortality | Log | Odds | |
| | | Rate | | Rate | Rank P- | Ratio | Multivariate |
| Analysis | N | (95% CI) | N | (95% CI) | value | (95% CI) | P-Value |
| Primary Analysis: | 28 | 0 (0.00- | 28 | 10.02 (2.73- | 0.0273 | 0.011 (0.0004- | 0.0061 |
| Matched on Age and Sex | | 5.05) | | 25.65) | | 0.28) | |
| Primary Analysis with | 28 | 1.55 (0.04- | 28 | 11.76 (3.82- | 0.0357 | 0.013 (0.0007- | 0.0029 |
| Extended Follow-Up | | 8.64) | | 27.45) | | 0.23) | |
| Matched on Age and Sex, | 25 | 0.00 (0.00- | 25 | 10.62 (2.89- | 0.0255 | 0.0072 (0.0003- | 0.0033 |
| Excluding 1980s Patients | | 5.37) | | 27.20) | | 0.19) | |
| Matched on Age and Sex, | 25 | 1.74 (0.04- | 25 | 10.43 (2.84- | 0.0899 | 0.023 (0.0016- | 0.0055 |
| Excluding 1980s Patients | | 9.71) | | 26.69) | | 0.33) | |
| with Extended Follow-Up | | | | | | | |
| Matched on Age | 32 | 0.00 (0.00- | 32 | 20.71 (8.32- | 0.0019 | 0.0071 (0.0004- | 0.0012 |
| | | 4.35) | | 42.66) | | 0.14) | |
| Full Cohort Comparison | 33 | 0.00 (0.00- | 34 | 12.08 (4.86- | 0.003 | 0.014 (0.0006- | 0.0065 |
| | | 4.24) | | 24.88) | | 0.30) | |
| Matched on Sex and | 28 | 0.00 (0.00- | 28 | 8.86 (2.41- | 0.0211 | 0.026 (0.0011- | 0.024 |
| Follow-Up Duration | | 5.03) | | 22.69) | | 0.62) | |
| Matched on Age and Sex | 28 | 0.00 (0.00- | 28 | 10.24 (2.79- | 0.0273 | Not Calculated | |
| with Unknown Onset | | 5.05) | | 26.22) | | | |
| Imputed at 0 | | | | | | | |
| Matched on Age and Sex | 28 | 1.55 (0.04- | 28 | 12.01 (3.90- | 0.0357 | | |
| with Unknown Onset | | 8.64) | | 28.03) | | | |
| Imputed at 0 with Extended | | | | | | | |
| Follow-Up | | | | | | | |
| Matched on Age and Sex | 31 | 0.00 (0.00- | 31 | 8.37 (2.28- | 0.0295 | | |
| with Replacement | | 4.46) | | 21.42) | | | |
| Matched on Age and Sex | 31 | 1.38 (0.03- | 31 | 9.93 (3.22- | 0.0443 | | |
| with Replacement with | | 7.70) | | 23.16) | | | |
| Extended Follow-Up | | | | | | | |

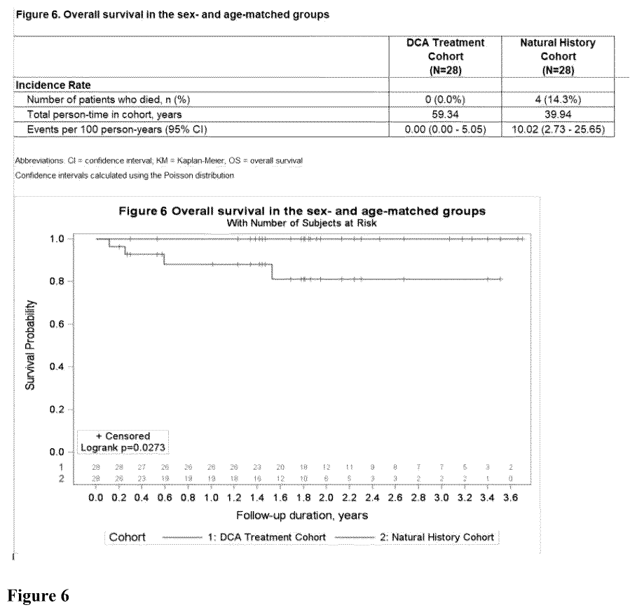

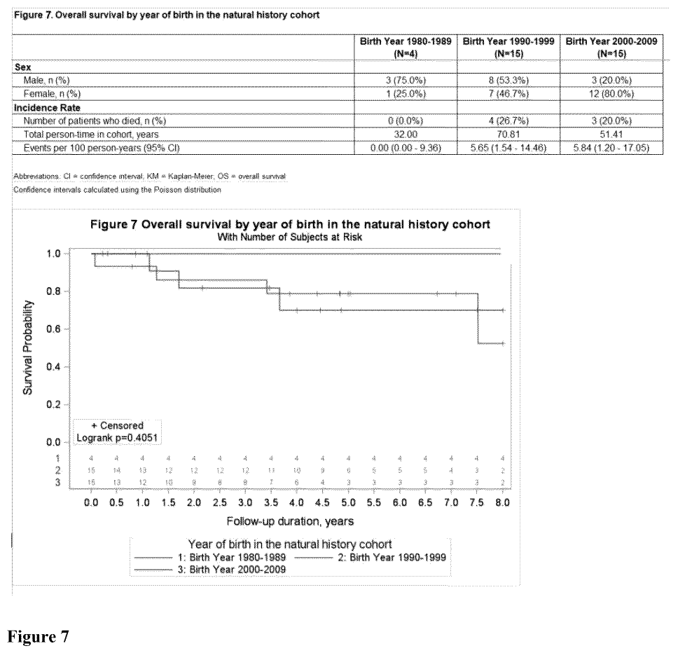

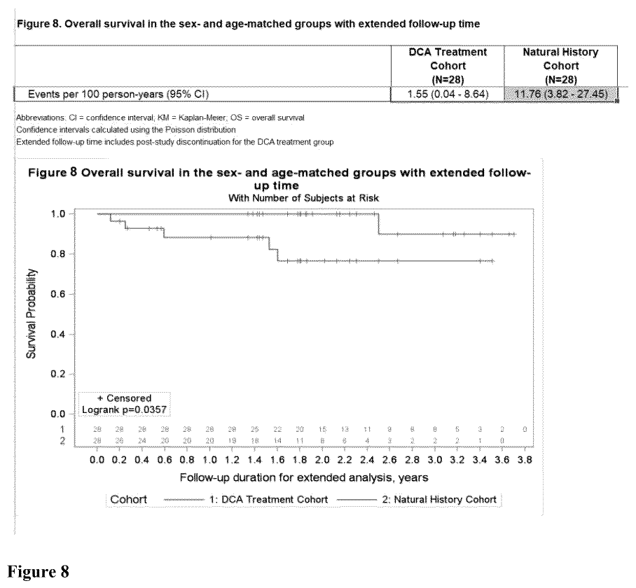

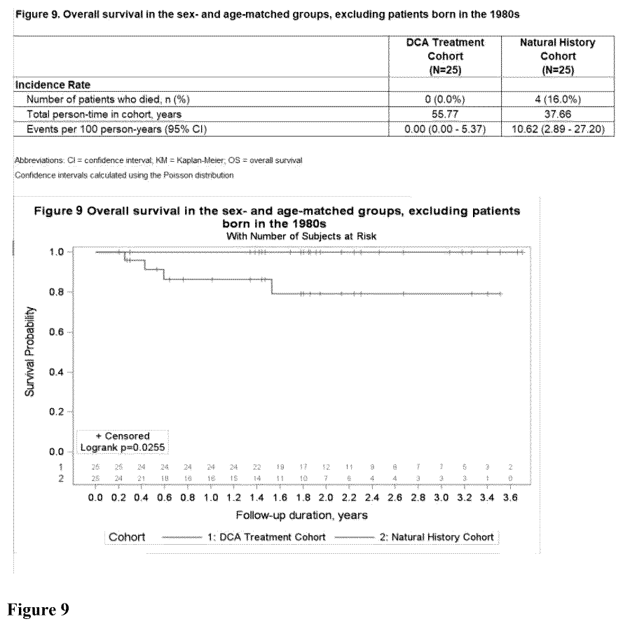

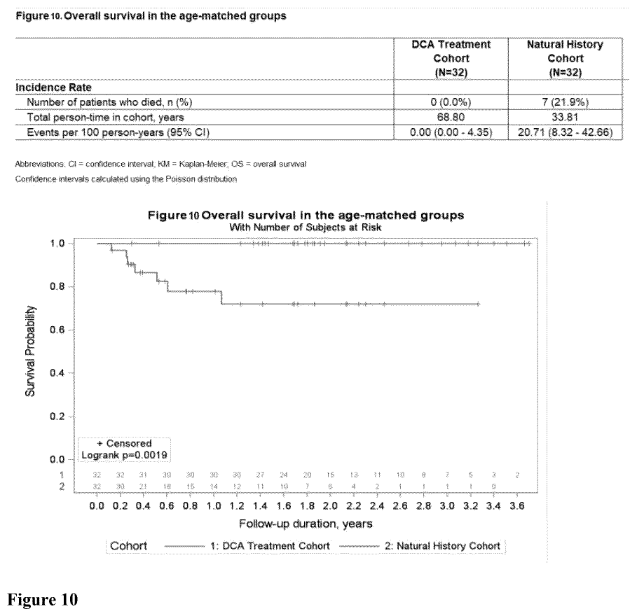

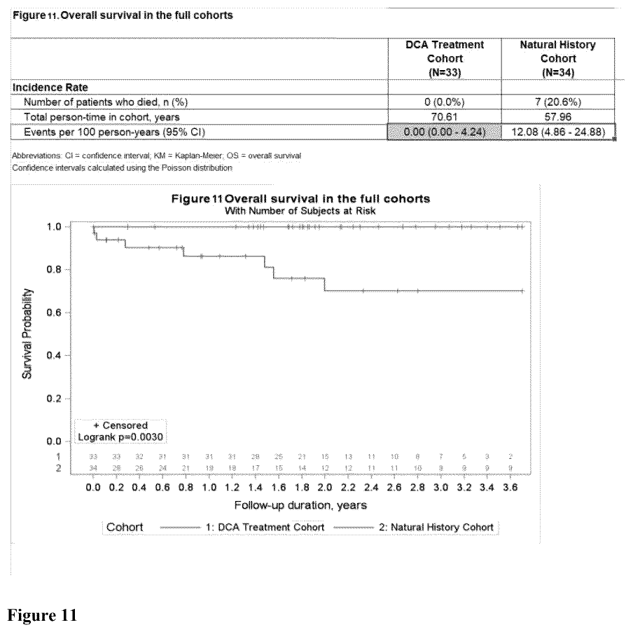

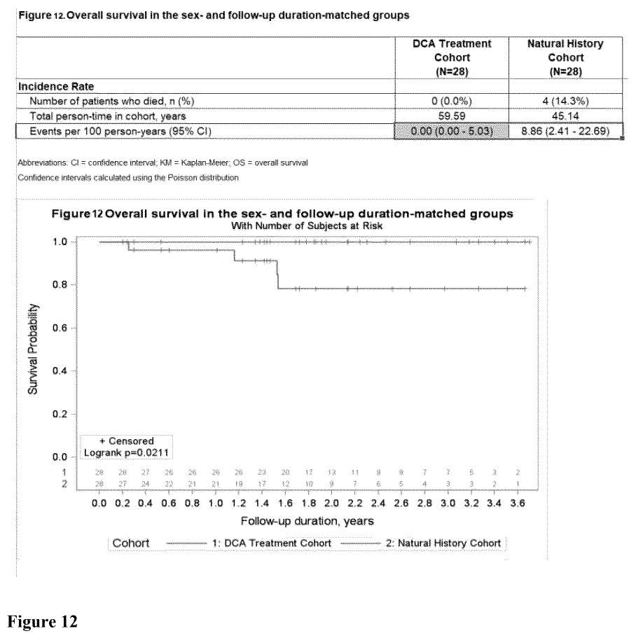

The results of the clinical studies are summarized in FIGS. 6-12.

The study results were surprising in a number of ways:
a. During the double-blind study, there were zero events of peripheral neuropathy. This was a highly unexpected outcome given the high level of variables related to metabolism of DCA. Given that there were no events, it was concerning that the dose of one or both of the groups was potentially too low to be clinically meaningful.
b. During the open-label phase, there was one event of neuropathy that was possibly related to therapy. This was an unusual presentation as it occurred after 18 months of exposure and was therefore inconsistent with the typically rapid onset events from prior studies. Neuropathy is also a potential symptom of PDCD, which may have been the cause.
c. There were zero deaths during the double-blind study and for patients that maintained chronic maintenance on therapy the extension study. This is also highly unexpected given the disease state and the age of the patients recruited (˜½ were between 6 and 24 months of age at the time of enrollment).
d. One patient discontinued DCA treatment for personal reasons after completing the double-blind portion of the study and continuing onto the open-label. 6 months following discontinuation of medication, the patient passed away.
e. There were zero dropouts for other reasons during the double-blind portion of the study. The investigators had anticipated a dropout rate of 20%, which would have been a substantial improvement on the previous rates of ˜44% mentioned earlier.

Although there was a suspicion that haplotype testing would reduce the risk of neuropathy and treatment with DCA may improve the course of the disease for some patients, it is highly unexpected that the haplotype testing had this large of an impact on the risk of peripheral neuropathy in this population without sacrificing efficacy given that the treatment response is exposure dependent. Someone learned in the art would have expected that the lack of adverse events would signal a potentially subtherapeutic effect of DCA in this population. It also did this without requiring changes in dose and across a wide range of ages in patients suffering from PDCD.

The invention presents a method of utilizing haplotype testing to identify a dose that optimizes the narrow therapeutic window of DCA and eliminates the risk of peripheral neuropathy while improving clinical outcomes, including increased survival for PDCD patients that chronically receive therapy.

Although the present invention has been described in considerable detail with regard to certain versions thereof, other versions are possible, and alterations, permutations and equivalents of the version shown will become apparent to those skilled in the art upon a reading of the specification and study of the drawings. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

### REFERENCES

Langaee et al., Pharmacogenet Genomics. 2015 May; 25(5): 239-245
Debrosse et al., Molecular Genetics and Metabolism 107 (2012) 394-402
Patel et al., Molecular Genetics and Metabolism 106 (2012) 385-394
Kaufmann et al., Neurology 2006; 66; 324-330
Stacpoole et al., Pediatrics Volume 121, Number 5, May 2008, e1223-e1228
Dunbar et al., Invest New Drugs, 2014; June; 32(3); 452-464
Michelakis et al., Sci. Transl. Med. 9; eaao4583 (2017) 25 Oct. 2017
Abdelmalak et al., Molecular Genetics and Metabolism 109 (2013) 139-143

What is claimed is:

1. A method of improving survival and minimizing treatment-limiting toxicities in dichloroacetate (DCA) treatment of patients with pyruvate dehydrogenase complex deficiency (PDCD) comprising: 1) identifying the DCA-metabolic haplotype of the patient, and 2) administering a DCA dose based on the DCA-metabolic haplotype.

2. The method of claim 1, wherein identifying the DCA-metabolic haplotype is performed by assaying for the GSTZ1 haplotype in the patient.

3. The method of claim 1, wherein the method reduces the likelihood of DCA-induced side effects to less than 10%.

4. The method of claim 3, wherein the method reduces the likelihood of DCA-induced side effects to less than 5%.

5. The method of claim 4, wherein the method reduces the likelihood of DCA-induced side effects to less than 2%.

6. The method of claim 1, wherein the method reduces the likelihood of PDCD-related mortality to less than 10%.

7. The method of claim 6, wherein the method reduces the likelihood of PDCD-related mortality to less than 5%.

8. The method of claim 7, wherein the method reduces the likelihood of PDCD-related mortality to less than 2%.

9. The method of claim 1, wherein the method reduces the odds ratio of death to less than 0.10%.

10. The method of claim 9, wherein the method reduces the odds ratio of death to less than 0.05%.
